(19) United States
(12) Patent Application Publication
(10) Pub. No.: US 2017/0348167 A1
(43) Pub. Date: Dec. 7, 2017

(54) ABSORBENT ARTICLE

(71) Applicant: OJI HOLDINGS CORPORATION, Tokyo (JP)

(72) Inventors: Izumi Tashiro, Tokyo (JP); Yoshihiro Takiyama, Tokyo (JP); Akira Sonoda, Tokyo (JP)

(21) Appl. No.: 15/534,202

(22) PCT Filed: Dec. 9, 2015
(86) PCT No.: PCT/JP2015/006134

§ 371 Date: Jun. 8, 2017

(30) Foreign Application Priority Data
Dec. 10, 2014 (JP) 2014-249967
Nov. 20, 2015 (JP) 2015-228188

(51) Int. Cl. A61F 13/539; A61F 13/49; A61F 13/511; A61F 13/514

(52) U.S. Cl. CPC A61F 13/539; A61F 13/514; A61F 13/49; A61F 13/511; A61F 2013/49098

(57) ABSTRACT

An absorbent article has a liquid-permeable front surface sheet, a liquid-impermeable back surface sheet, and an absorber disposed between the front surface sheet and the back surface sheet. The absorbent article includes a groove portion entirely or partly composed of a recessed portion, the recessed portion being formed on the front surface sheet side of the absorbent article by integrally joining the front surface sheet and the absorber together, and a flat portion enclosed by the groove portion. A diameter of a maximum inscribed circle inscribed in the flat portion is at least five times and at most 20 times as large as a width of the groove portion along a radial direction of the maximum inscribed circle.

### TECHNICAL FIELD

The present invention relates to an absorbent article, and in particular, to a disposable diaper.

### BACKGROUND ART

Absorbent articles such as sanitary napkins and disposable diapers include an absorber to absorb most of a body fluid such as discharged urine (hereinafter also referred to as a “body fluid”). Therefore, after an absorbent article absorbs the body fluid, a space between the skin and the absorbent article is humid, causing the skin to feel sticky. Conventional absorbent articles are designed to suppress a possible sticky feeling resulting from absorption of the body fluid. For example, regular fine recesses and protrusions such as a staggered pattern are formed on a front surface sheet of an absorbent article that comes into direct contact with the skin, as disclosed in PTL 1. Furthermore, PTL 2 discloses that a front surface sheet, along with an absorbent, is compressed into a recessed and protruding shape. As described above, the front surface sheet (or the front surface sheet and the absorbent) is provided with recesses and protrusions to allow tips of protruding portions to contact the skin. Thus, this configuration reduces the area of a part of the front surface sheet that contacts the skin, suppressing the sticky feeling, compared to a configuration with no recesses or no protrusions.

### CITATION LIST

### Patent Literature

PTL 1 Japanese Patent Laid-Open No. 2003-275239

PTL 2 Japanese Patent Laid-Open No. 2012-213488

### SUMMARY OF INVENTION

### Technical Problem

For example, a disposable diaper needs to have an absorbing capacity sufficient to reliably absorb an amount of urine corresponding to several times of urination. To enhance the absorbing capacity, many absorbers are formed of a mixture of pulp and a super absorbent polymer (hereinafter also referred to as a “SAP”). Thus, in a case where the absorbent article described in PTL 1 absorbs the body fluid, the absorber bulges significantly due to the effect of the SAP. The bulge of the absorber itself offsets the recesses and protrusions provided on the front surface sheet, which thus produce little effect.

Furthermore, in the absorbent article described in PTL 2, the recesses and protrusions are formed by compressing the front surface sheet and the absorbent article. However, the recesses and the protrusions still need to be improved in terms of dimensions in order to suppress the sticky feeling.

In view of these problems, an object of the present invention is to provide an absorbent article that restrains the skin from feeling sticky in a case where the skin comes into contact with an absorber having absorbed a body fluid.

### Solution to Problem

An absorbent article in the present invention has a liquid-permeable front surface sheet, a liquid-impermeable back surface sheet, and an absorber disposed between the front surface sheet and the back surface sheet. The absorbent article in the present invention has a groove portion entirely or partly composed of a recessed portion, the recessed portion being formed on the front surface sheet side of the absorbent article by integrally joining the front surface sheet and the absorber together, and a flat portion enclosed by the groove portion. A diameter of a maximum inscribed circle inscribed in the flat portion is at least five times and at most 20 times as large as a width of the groove portion along a radial direction of the maximum inscribed circle.

Furthermore, the recessed portion may enclose at least 50% of an outer periphery of the flat portion.

The recessed portion is formed by compressing the front surface sheet and the absorber together, and a depth of the recessed portion is at least half of a thickness of each of the flat portion.

Additionally, the groove portions may be shaped like a lattice.

Furthermore, an absorbent article in the present invention has a liquid-permeable front surface sheet, a liquid-impermeable back surface sheet, and an absorber disposed between the front surface sheet and the back surface sheet. The absorbent article in the present invention has a groove portion entirely or partly composed of a recessed portion, the recessed portion being formed and arranged on the front surface sheet side of the absorbent article by integrally joining the front surface sheet and the absorber together and a flat portion positioned between the groove portions. A diameter of a maximum inscribed circle inscribed in the flat portion sandwiched between the two adjacent groove portions is at least five times and at most 20 times as large as a width of the groove portion along a radial direction of the maximum inscribed circle.

Additionally, the groove portions may be arranged at regular intervals.

Furthermore, an amount of SAP per unit area in the absorber may be at least 0.01 g/cm².

Additionally, the absorbent article may be a disposable diaper.

### Advantageous Effects of Invention

The absorbent article in the present invention includes the flat portion having at least a given area, and thus, the flat portion allows a body fluid to be quickly absorbed. Furthermore, in the present invention, the absorbent article does not perfectly fit a curved surface of a wearer's body but locally forms a gap between the absorbent article and the curved surface. Consequently, even after absorbing the body fluid, the absorbent article is likely to have a gap between the absorbent article and the skin and is thus unlikely to cause the wearer to feel sticky.

### BRIEF DESCRIPTION OF THE DRAWINGS

FIG. 1 is a perspective view depicting the appearance of Embodiment 1 in which an absorbent article according to the present invention is applied to a pull-up type disposable diaper;

FIG. 2 is a perspective view depicting a lower body wearing the diaper depicted in FIG. 1, as seen from the back side;

FIG. 3 is a perspective view illustrating that the diaper depicted in FIG. 1 has been disassembled;

FIG. 4 is a partial top view of a groin portion of the diaper depicted in FIG. 1, as seen from a top sheet side;

FIG. 5 is a sectional view taken along a V-V direction of the groin portion of the diaper depicted in FIG. 4;

FIG. 6 is a schematic diagram illustrating that after an absorbing portion of the diaper in Embodiment 1 absorbs a body fluid, the diaper contacts a curved surface of a wearer's body with gaps kept between the diaper and the curved surface;

FIG. 7 is an enlarged partial top view of a front surface of the absorbing portion of the diaper in Embodiment 1;

FIG. 8 is a partial top view illustrating an example in which compressed portions are arranged at intervals to form a recessed portion;

FIG. 9 is a sectional view taken along an IX-IX direction in FIG. 8;

FIG. 10 is a sectional view taken along an X-X direction in FIG. 8;

FIG. 11 is a partial top view illustrating an example of a flat portion pattern formed by arranging recessed portions in the absorbing portion at regular intervals;

FIG. 12 is a partial top view illustrating an example of a flat portion pattern formed by arranging wavelike recessed portions in the absorbing portion;

FIG. 13 is a partial top view illustrating an example of a flat portion pattern formed by arranging circular recessed portions in the absorbing portion at regular intervals; and

FIG. 14 is a partial top view illustrating an example of a flat portion pattern formed by arranging, in the absorbing portion, discontinuous recessed portions shaped like regular hexagons.

### DESCRIPTION OF EMBODIMENTS

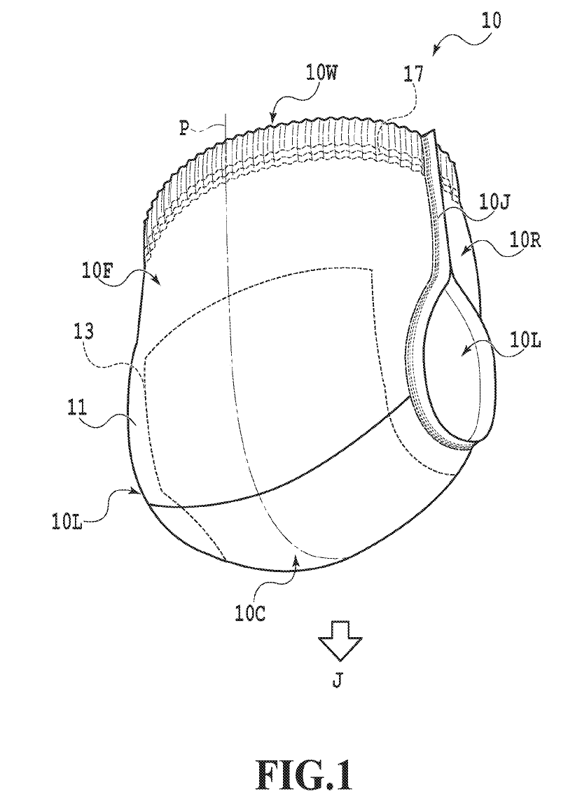

An embodiment of an absorbent article according to the present invention will be described in detail with reference to FIGS. 1 to 14. However, the present invention is not limited to the aspects of the present embodiment.

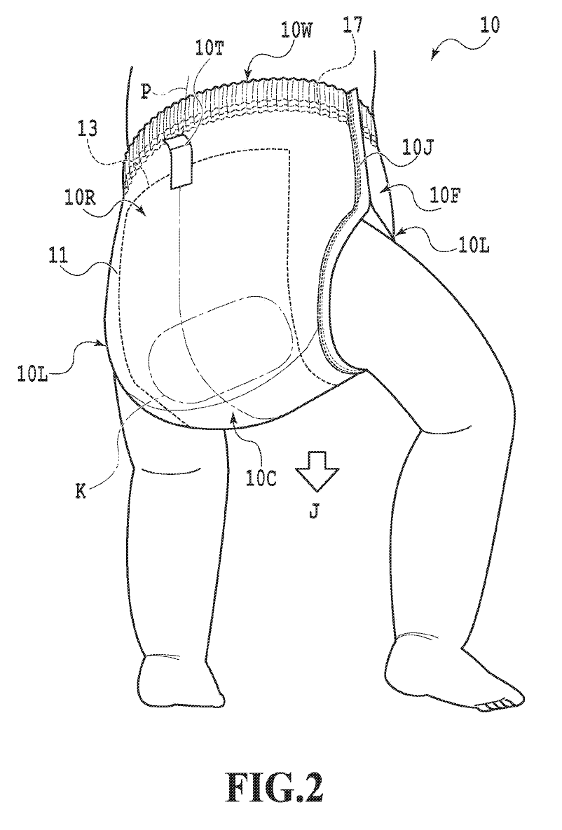

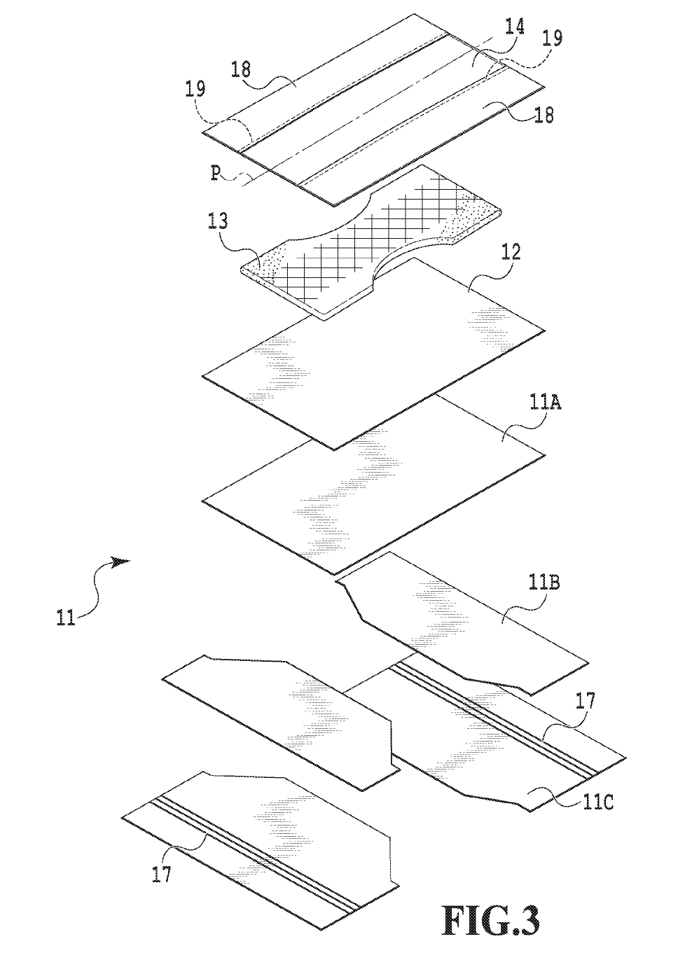

FIG. 1 depicts a perspective view of the appearance of Embodiment 1 in which the present invention is applicable to a pull-up type disposable diaper (hereinafter simply referred to as a “diaper”) 10 as seen from a front side, and FIG. 2 is a perspective view of the appearance as seen from a back side. FIG. 2 depicts the lower body of an infant wearing the diaper 10 as seen from the back side. Moreover, FIG. 3 illustrates that the diaper 10 depicted in FIG. 1 and FIG. 2 is in a divided state where the diaper 10 is torn and unfolded.

The diaper 10 in the present embodiment has a front body area 10F, a back body area 10R, and a groin area 10C connecting the front body area 10F and the back body area 10R together. Furthermore, a waist opening 10W is formed such that a wearer's waist portion is surrounded by the front body area 10F and the back body area 10R. A part of the front body area 10F, a part of the back body area 10R, and the groin area 10C form a lateral pair of leg openings 10L surrounding the thigh portions of the wearer's legs.

As depicted in FIG. 2, in a case where the diaper 10 is worn by the wearer, the front body area 10F is positioned the wearer's abdominal side, whereas the back body area 10R is positioned on the wearer's back side. The groin area 10C covers the wearer's groin, and the wearer's legs are inserted through the lateral pair of leg openings 10L, respectively. Therefore, the leg openings 10L are each located at any position between the wearer's groin and the vicinity of the corresponding thigh.

Furthermore, on the back body area 10R of the diaper 10, a disposal tape 10T is provided that is used to tape the rolled diaper 10 in a case where the diaper 10 is disposed of.

Now, a virtual line P will be described that extends in a direction along the central axis of the body in a case where the diaper 10 is worn in an appropriate orientation. As depicted in FIG. 1 and FIG. 2, the virtual line P extends along the central axis of the body, and at a central portion of the diaper 10, extends through the groin portion from the abdominal side toward the back side. Specifically, in a case where, for example, the waist side of the diaper 10 is designated as an upper side and the groin side is designated as a lower side, the virtual line P extends along the front surface of the diaper 10 and in an up-down direction and extends through the groin portion and also in the up-down direction on the back side. Positional relations among the areas of the diaper 10 will be described using the virtual line P as required, as follows.

Now, as depicted in FIG. 3, the diaper 10 in the present embodiment is formed by laying a cover sheet 11, a back sheet (back surface sheet) 12, an absorber 13, a top sheet (front surface sheet) 14, and a pair of side sheets 18 on top of one another in this order from the outside and joining these components together; the top sheet 14 contacts the wearer's skin, and the side sheets 18 allow raised gathering to be formed. Moreover, the cover sheet 11 is divided into a pad cover sheet 11A placed under the back sheet 12, an inner cover sheet 11B, and an outer cover sheet 11C all of which are formed of a thin nonwoven cloth so as to have a smooth feel. The inner cover sheet 11B and the outer cover sheet 11C, which define the front body area 10F and the back body area 10R of the diaper 10, are joined together such that laterally opposite edges of the inner cover sheet 11B face the corresponding laterally opposite ends of the outer cover sheet 11C, thus forming closed portions 10J. Consequently, the above-described waist opening 10W is defined. The inner cover sheet 11B and the outer cover sheet 11C are each divided into two pieces at the groin area 10C portion so as to expose the pad cover sheet 11A at the groin portion. Therefore, the lateral pair of leg openings 10L, which surrounds the thigh portions of the respective legs, is defined by ends of the pad cover sheet 11A and ends of the closed portions 10J. The back sheet 12, which is impermeable to liquids, is joined to the pad cover sheet 11A. The above-described absorber 13 is disposed between the back sheet 12 and the top sheet 14, which is permeable to liquids. The top sheet 14 is joined to the back sheet 12 via the absorber 13. The pair of side sheets 18 is joined to the top sheet 14. Rubber threads 19 are stretched and joined to the pair of side sheets 18 at edges thereof in order to allow raised gathering to be formed. The inner cover sheet 11B and the outer cover sheet 11C may each be an integral sheet that is not discontinuous at the groin portion. The gathering may be formed at the leg openings 10L by the rubber threads.

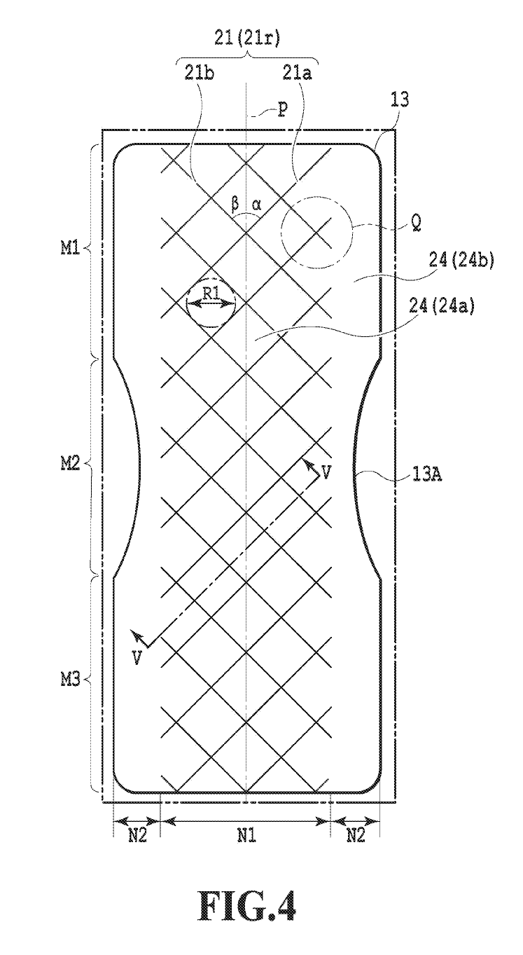

FIG. 4 is a partial top view of the groin portion of the diaper 10 as seen from the top sheet 14.

The absorber 13 in the present embodiment, positioned under the top sheet 14, is mainly formed of pulp and SAP. The ratio of the amount of SAP to the amount of pulp is preferably 1:0.8 to 1:1.5. Furthermore, the amount of SAP per unit area in the absorber 13 is preferably 0.01 g/cm². In the absorber 13 in the present embodiment, the amount of pulp per unit area is 0.017 g/cm², and the amount of SAP per unit area is 0.023 g/cm².

The absorber 13 has an elongate shape so as to span the front body, the groin, and the back body. The absorber 13 is partitioned into three portions, a front body portion M1, a groin portion M2, and a back body portion M3 as depicted in FIG. 4. In the groin portion M2, cutout portions 13A are formed that are shaped like circular arcs conforming to the lateral pair of leg openings 10L surrounding the thigh portions of the respective legs. The cutout portions 13A may be omitted depending on the size of the absorber 13. Furthermore, the absorber 13 in the present embodiment is provided with the cutout portions 13A and shaped like a hourglass that is narrower at a central portion thereof than at each of a front and a back ends thereof. However, the shape of the absorber in the present invention is not limited to this. In a case where a direction from the front body portion toward the back body portion is designated as a front-back (up-down) direction and a direction orthogonal to the front-back direction is designated as a lateral direction, the absorber in the present invention may have various shapes as follows. For example, corners of the front and back ends (upper and lower ends) may be cut round or the absorber may be shaped like an ellipse that extends in the front-back (up-down) direction, a circle, a rectangle in which a front-back (up-down) length is different from a lateral length, or a square in which the front-back (up-down) length is the same as the lateral length.

An absorbing portion of the diaper 10 including the top sheet 14 and the absorber 13 (hereinafter simply referred to as an “absorbing portion”) includes a groove portion 21 and a flat portion 24. In the descriptions in the specification and the claims, the “groove portion” refers to a portion entirely or partly composed of a recessed portion. In the descriptions in the specification and the claims, the “recessed portion” refers to that part of the absorbing portion of the absorbent article (in the present invention, the diaper 10) which has a thickness that is at most half the thickness of the uncompressed absorbing portion. Furthermore, in the descriptions in the specification and the claims, the “flat portion” refers to that part of the absorbing portion of the absorbent article which corresponds to the entire absorbing portion except for the groove portion. The flat portion may be shaped like a chevron, a wave, a trapezoid, a rectangle, or the like. In particular, for the chevron shape, a peak portion of the chevron shape contacts the skin, whereas a base portion of the chevron shape is unlikely to contact the skin.

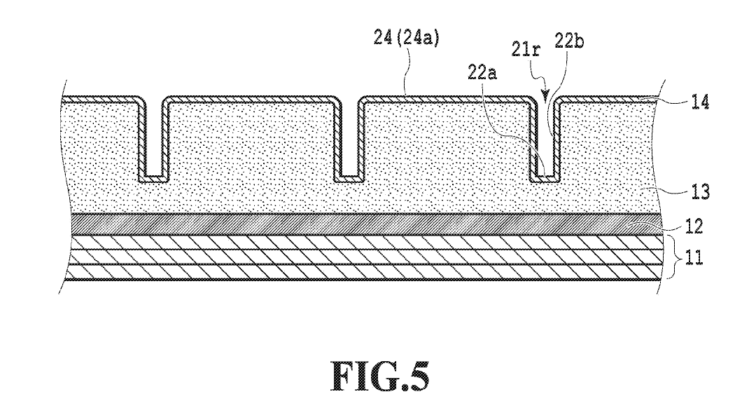

The flat portion 24 has two types of flat portions, a first flat portion 24a that is enclosed by the groove portion 21 and a second flat portion 24b that is not enclosed by the groove portion 21. In the present embodiment, as depicted in FIG. 4, all of the groove portions 21 are composed of the recessed portion 21r, and thus, the flat portion 24 has the first flat portion 24a that is enclosed by the recessed portion 21r and the second flat portion 24b that is not enclosed by the recessed portion 21r. The diaper 10 according to the present embodiment includes at least the first flat portion 24a. In the present embodiment, as depicted in FIG. 4 and FIG. 5 described below, the absorbing portion is provided with the regularly arranged recessed portions 21r by being compressed from a front surface of the top sheet 14 toward the absorber 13. Thus, a surface that contacts the skin is partitioned into a plurality of areas.

In other words, the absorbing portion, which is originally generally flat, is, for example, partly compressed and recessed by embossing to form the recessed portion 21r. The recessed portions 21r serve to divide the absorbing portion into a plurality of the flat portions 24. Therefore, all the adjacent first flat portions 24a are separated from each other by the corresponding recessed portion 21r such that the first flat portions 24a and the recessed portions 21r are regularly arranged on the front surface of the absorbing portion.

In the present embodiment, the recessed portions 21r are not formed up to each end of the absorbing portion of the diaper 10 in a width direction (the lateral direction in FIG. 4) but are formed like bands in a central portion of the diaper 10 along a longitudinal direction (the up-down direction in FIG. 4). A recessed portion forming area N1 is defined as an area of the absorbing portion where the recessed portions 21r are formed, and a recessed portion non-forming area N2 is defined as areas located at widthwise opposite ends of the absorbing portion where no recessed portions 21r are formed. As described below, the configuration with the recessed portion 21r is more effective than a configuration with no recessed portion 21r so long as the recessed portion 21r are formed at least in the central portion of the diaper 10. The present embodiment includes the recessed portion non-forming area N2. However, the present invention is not limited to this form. Of course, the recessed portion 21r may be formed up to each end of the absorber 13. Therefore, in the present invention, the recessed portion non-forming area N2 may be omitted.

FIG. 5 is a sectional view of the groin portion of the diaper depicted in FIG. 4, the view being taken along a V-V direction. FIG. 5 depicts a section of the recessed portions 21r extending like diagonal lattices. An adhesive is applied to a surface of the top sheet 14 that contacts the absorber 13. The top sheet 14 and the absorber 13 are compressed by embossing to form the recessed portions 21r. Consequently, the top sheet 14 and the absorber 13 are engaged with each other all over the recessed portion 21r spanning a bottom surface (bottom wall) 22a of the recessed portion 21r and a wall surface (side wall) 22b of the recessed portion 21r, and are thus integrally joined together. An example of the thickness of the top sheet 14 and the absorber 13 at the bottom portion of the recessed portion 21r (the thickness of the absorbing portion) is one-sixth of the thickness of the uncompressed absorbing portion. In this case, the depth reaches approximately 80% of the thickness of the top sheet 14 and the absorber 13 that are laminated to each other and is larger than in a case where the recesses and protrusions are formed only on the top sheet 14. The magnitude of compression of the absorber 13 preferably corresponds to approximately half to one-seventh of the thickness of the uncompressed absorber 13. More preferably, the magnitude of compression of the absorber 13 corresponds to approximately quarter to one-seventh of the thickness of the uncompressed absorber 13. Furthermore, the depth of the recessed portion 21r is preferably six-sevenths to half and more preferably six-sevenths to three-fourths of the thickness of the flat portion 24. The present invention is applicable to an absorber having a thickness that is not constant (that varies) before embossing.

For the diaper, for example, a large amount of urine discharged at a time needs to be instantaneously absorbed by the absorber 13. On the other hand, the recessed portion 21r, where the top sheet 14 and the absorber 13 are compressed, is less likely to absorb the body fluid than an area where the absorber 13 is not compressed, because the absorber 13 is compressed in the recessed portion 21r. The main absorbing area is the flat portion 24 that is not compressed. Therefore, the flat portion 24 needs to have an area sufficient to allow the body fluid to be instantaneously absorbed and held.

For the flat portion 24 in the present invention, this area is determined based on observations described below.

As depicted in FIG. 1 and FIG. 2, in a case where the diaper 10 is worn by the wearer, the absorber 13 is positioned so as to extend from the abdominal side toward the back side and to cover the entire groin portion. As seen in FIG. 2, while the wearer is standing, the diaper 10 is deflected and droop down (in a direction depicted by arrow J) such that the central portion of the groin area 10C corresponds to the peak of the deflected shape. This prevents the wearer from coming into close contact with the central portion of the groin portion. The reason why this configuration is used is as follows. In a case where the absorber 13 bulged with the body fluid comes into close contact with the groin, the groin is likely to be steamed. Thus, a certain amount of space is preferably provided between the groin and the diaper 10. On the other hand, a buttocks area K that is continuous with the groin area 10C and that faces a round buttocks portion of the body is a portion that is likely to come into close contact with the skin in conjunction with an operation of pulling up the diaper 10.

In a case where the wearer discharges the body fluid while lying on the wearer's back, the body fluid is likely to flow not only through the groin portion 10C but also through the back body area 10R. In particular, while the wearer is lying on the wearer's back, the buttocks area K is located at the lowest position and is thus an area into which the body fluid is likely to flow. Also in a case where the wearer sits up, the buttocks area K is located at the lowest position and is thus an area into which the body fluid is likely to flow.

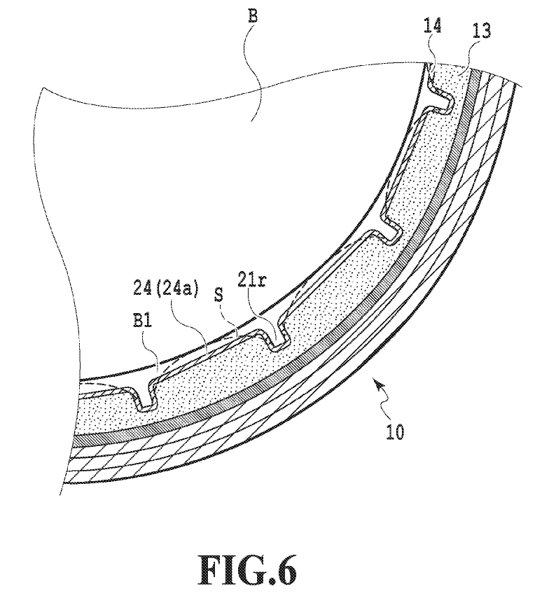

FIG. 6 is a schematic diagram illustrating that after the absorbing portion of the diaper 10 in Embodiment 1 absorbs the body fluid, the diaper 10 contacts the curved surface of the wearer's body with gaps kept between the diaper and the curved surface.

As depicted in FIG. 6, in the diaper 10 in the present embodiment, the absorbing portion is provided with the first flat portions 24 having above certain sizes and the recessed portions 21r that separates the adjacent first flat portions 24a from each other. Therefore, in a case where the diaper 10 is positioned along the curved surface of the wearer's body B, the recessed portions 21r appear at regular intervals such that the recessed portions 21r arranged at the regular intervals form gaps between the diaper 10 and the skin.

In addition, the recessed portion 21r is likely to fold in the width direction of the recessed portion 21r at portions where both the top sheet 14 and the absorber 13 are compressed. That is, the absorbing portion folds at the bottom surfaces 22a of the recessed portion 21r so as to form valley folds or peak folds, and thus provides multiple surfaces arranged along the body so as to approximate the curved surface of the body with reference to the surface width of each first flat portion 24a. Consequently, each of the first flat portions 24a is arranged along the curve of the body B, and thus, the absorbing portion does not perfectly fit the circular arc of the curved surface but a space B1 is formed between the skin and the absorbing portion facing the skin.

Furthermore, even after the absorbing portion absorbs the body fluid, each first flat portion 24a bulges like a small dome such that the center of the first flat portion 24a corresponds to the peak of the dome. As depicted by a dotted line S in FIG. 6, the first flat portion 24a has a certain size and thus bulges like a dome (so as to have a vault-like cross section). Therefore, even in a case where the first flat portion 24a bulges and comes into contact with the skin, the contact area is not the entire area of the first flat portion 24a but corresponds to the peak of the dome. This reduces the area over which the first flat portion 24a contacts the skin, suppressing a possible sticky feeling. Furthermore, gaps are present in the areas other than the contact portions, thus providing ventilation and enabling the skin to be restrained from being steamed or getting diaper rash.

As described above, in order to suppress the sticky feeling, the first flat portion 24a needs to have a certain size. Specifically, the optimal size of the first flat portion 24a is determined based on the diameter of the maximum inscribed circle inscribed in the first flat portion 24a enclosed by the recessed portion 21r (a groove portion 21) and the width of the recessed portion 21r (a groove portion 21) along a radial direction of the maximum inscribed circle. Here, the diameter of the maximum inscribed circle is denoted by R1, and the above-described width of the recessed portion 21r is denoted by L1.

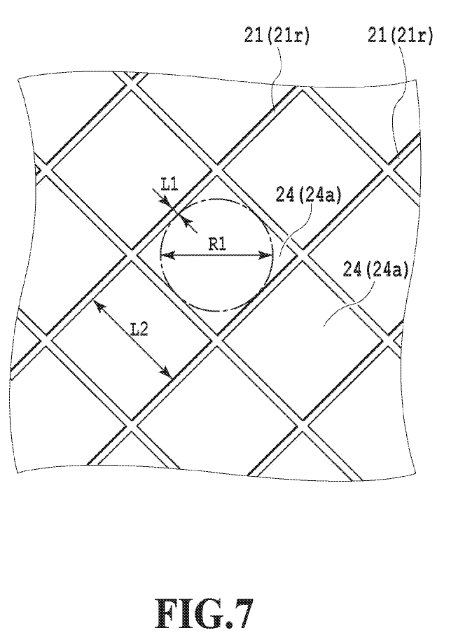

As depicted in FIG. 7, the width L1 of the recessed portion 21r in the present embodiment is approximately 2 mm. With the feel taken into account, the width L1 of the recessed portion 21r is preferably 1 mm or more and 5 mm or less. Furthermore, in the present embodiment, as depicted in FIG. 7, the recessed portion 21r enclose each first flat portion 24a so as to shape the first flat portion 24a like a square. Here, the length of each side of the square of the first flat portion 24a enclosed by the recessed portion 21r is denoted by L2. In the present embodiment, the length L2 is approximately 30 mm. Therefore, the diameter R1 of the maximum inscribed circle inscribed in the square is also approximately 30 mm. The size of the flat portion 24 is preferably such that 5 mm≦L2≦40 mm and 5 mm≦R1≦40 mm. Here, the diameter R1 of the maximum inscribed circle is preferably at least five times and at most 20 times as large as the width L1 of the recessed portion 21r and more preferably at least 10 times and at most 20 times as large as the width L1. In the present embodiment, the diameter R1 is approximately 15 times as large as the width L1.

In a case where the diameter R1 of the maximum inscribed circle is less than five times as large as the width L1 of the recessed portion 21r, the first flat portions 24a themselves are small in area and are not very effectively arranged along the curved surface of the body while maintaining spaces between the absorbing portion and the body. Furthermore, even in a case where the first flat portion 24a has a certain size, in a case where the width L1 of the recessed portion 21r is substantially large with respect to the first flat portion 24a, the area of the first flat portions 24a accounts for a low rate of the area of the entire absorbing portion. This causes the feel of the absorbing portion to be impaired and is not preferable. Furthermore, since the main absorbing area is the flat portion 24, an increase in the area of the recessed portion 21r reduces the amount of body fluid that can be absorbed. This is not efficient.

On the other hand, in a case where the diameter R1 of the maximum inscribed circle is more than 20 times as large as the width L1 of the recessed portion 21r, the interval between the recessed portions 21r is excessively large, and the first flat portions 24a themselves fit the curved surface of the body and closely contact the skin.

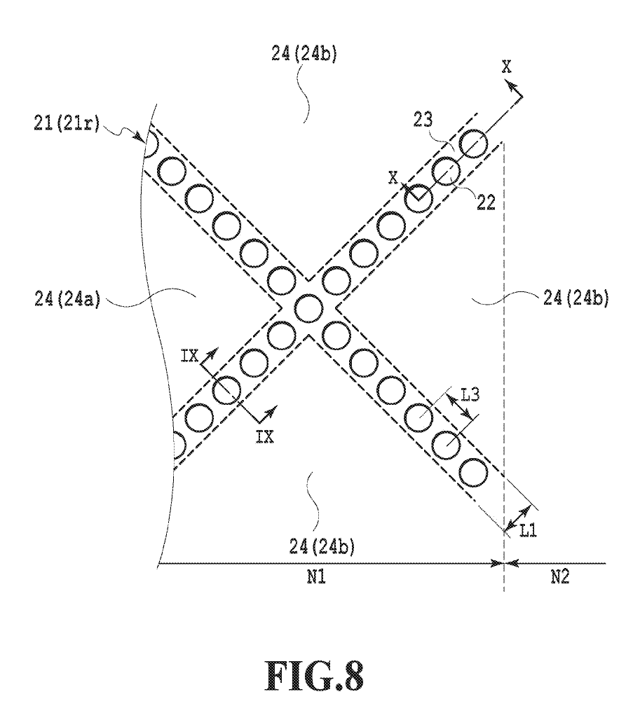

FIG. 8 illustrates a variation of Embodiment 1 of the present invention and illustrates an example in which the recessed portion 21r is formed of an emboss pattern with a plurality of dots within a range Q that strides over the recessed portion forming area N1 and the recessed portion non-forming area N2. The range Q is depicted by an alternate long and short dash line. In the present variation, all of the groove portions 21 are composed of the recessed portion 21r as is the case with Embodiment 1. The front surface of the top sheet 14 is compressed toward the absorber 13 using the dot-like emboss pattern to place compressed portions 22 at intervals. The compressed portions 22 are arranged at certain intervals to lower portions of the absorber 13 located near the compressed portions 22, resulting in depressed portions 23 that are recessed with respect to the flat portion 24. Therefore, the compressed portions 22 are formed contiguously or at intervals to form the recessed portions 21r each including the compressed portion 22 and the depressed portion 23.

The width L1 of the recessed portion 21r is preferably 1 mm≦L1≦5 mm as is the case with Embodiment 1, and a center distance L3 between the adjacent compressed portions 22 is preferably 1 mm≦L3≦8 mm. In the present variation, the width L1 is approximately 4 mm, and the distance L3 is approximately 6 mm.

In a case where the thickness of the uncompressed portion of the absorber 13 located in the center of the flat portion 24 is 3 to 3.6 mm and the density of the uncompressed portion is 0.16 g/cm³, the thickness of the absorber 13 is 0.5 to 0.6 mm and the density of the absorber 13 is 0.96 g/cm³ at each compressed portion 22, and the thickness of the absorber 13 is 1 to 1.2 mm and the density of the absorber 13 is 0.48 g/cm³ at each depressed portion 23. As described above, in a case where the difference in density between the compressed portion 22 of the recessed portion 21r and that portion of the absorber 13 located in the center of the flat portion 24 which corresponds to the main absorbing area is such that the density of the compressed portion 22 is approximately six times as high as the density of the portion of the absorber 13, the arrangement interval between the recessed portions 21r (or the length L2) is desirably adjusted so as to set the difference in density between the compressed portion 22 and the depressed portion 23 of the recessed portion 21r such that the density of the compressed portion 22 is approximately twice to three times as high as the density of the depressed portion 23.

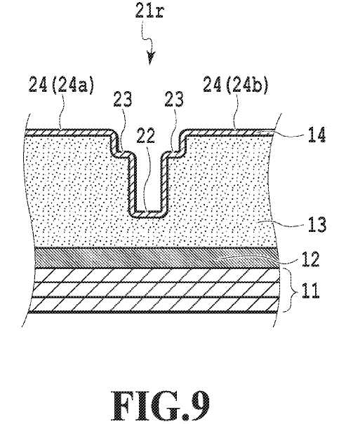

FIG. 9 is a sectional view taken along an IX-IX direction in FIG. 8.

As depicted in FIG. 9, embossing is performed by interposing an adhesive between the top sheet 14 and the absorber 13 and compressing both the top sheet 14 and the absorber 13 from the front surface of the top sheet 14 as is the case with the diaper depicted in FIG. 5.

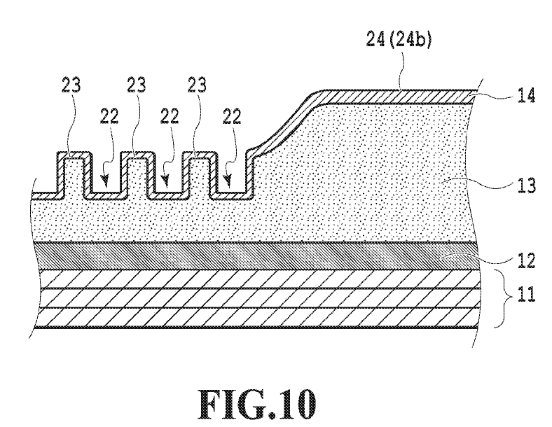

FIG. 10 is a sectional view taken along an X-X direction in FIG. 8.

As depicted in FIG. 10, the depressed portions 23 are positioned higher than the compressed portions 22 but lower than the flat portion 24 (the second flat portion 24b in FIG. 10) and the recessed portion non-forming area N2. Therefore, the compressed portions 22 are formed at predetermined intervals to allow obliquely extending recessed portions 21r as is the case with continuous embossing.

As seen back in FIG. 4, the recessed portion 21r having a recessed shape in the present embodiment includes first recessed portions 21a extending so as to incline toward a first side (in FIG. 4, toward the right) in the width direction of the absorber 13 and a plurality of second recessed portions 21b extending so as to incline toward a second side (in FIG. 4, toward the left) in the width direction of the absorber 13. The virtual line P is positioned on the absorber 13 so as to extend from an upper end of a front body portion M1 toward a lower end of a back body portion M3. Specifically, as depicted in FIG. 4, in a case where the absorber 13 has an elongate shape, the virtual line P extends in a longitudinal direction. Each recessed portion 21r extends so as to incline with respect to the virtual line P. That is, the first recessed portion 21a is inclined at an angle α to the first side and the second recessed portion 21b is inclined at an angle β to the second side, with respect to a longitudinal direction of the absorber 13, for example, a direction extending along sides of the absorber 13 in a case where the virtual line P is set as an axis. The angle α may be the same as or different from the angle β. Furthermore, in FIG. 4, the plurality of first recessed portions 21a are inclined at the same angle and arranged at regular intervals. That is, the first recessed portions 21a are arranged parallel to one another and at regular intervals. However, the present invention is not limited to this but includes a configuration in which the interval between the recessed portions 21r varies and a configuration in which the inclination angle varies among the recessed portions 21r. This also applies to the second recessed portion 21b. In the present embodiment, the “maximum inscribed circle” is a perfect circle. However, the present invention includes an ellipse. In a case where the maximum inscribed circle is an ellipse, the interval between the recessed portions 21r maybe varied (may be irregular). The above-described R1 for the case where the maximum inscribed circle is an ellipse is determined to be equal to the average of a major axis and a minor axis of the ellipse. The minor axis is preferably at least half of the major axis. The minor axis is set within this range to allow the adjacent recessed portions 21r to be appropriately spaced from each other and to allow the flat portion 24 to be provided with a sufficient area. The body fluid (for example, urine) flowing through the adjacent recessed portion 21r can be quickly absorbed by the flat portion 24.

### Other Embodiments

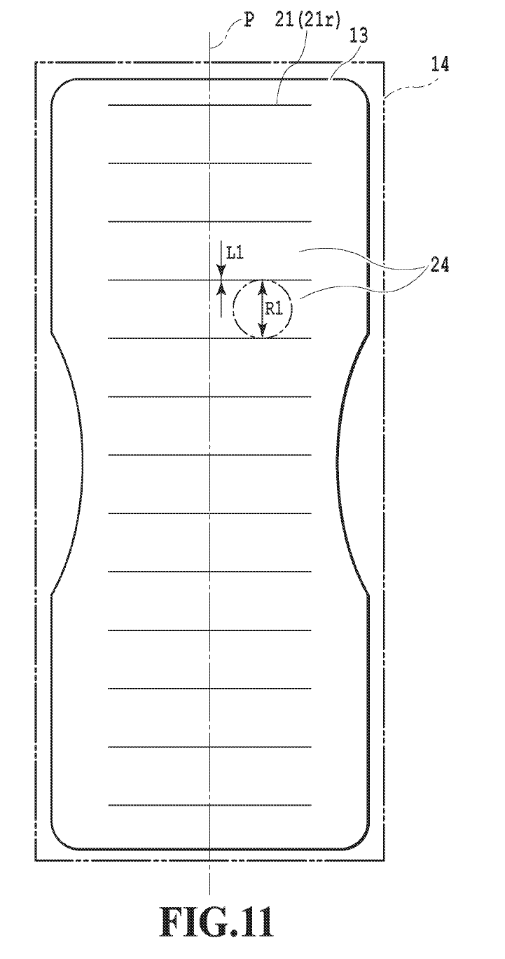

The flat portion pattern of the absorbing portion is not limited to an oblique arrangement of squares depicted in FIG. 4 and described in Embodiment 1 of the present invention. The following patterns are also possible. Other embodiments will be described below using FIGS. 11 to 14. For the elements of the diaper 10 in this embodiment, those which have functions similar to the functions in Embodiment 1 are denoted by the same reference numerals and will not be described below.

FIG. 11 is a partial top view illustrating an example of a flat portion pattern formed by arranging the recessed portions 21r (groove portions 21) at regular intervals in the absorbing portion. In the example illustrated in FIG. 11, the recessed portions 21r extend orthogonally to the virtual line P to partition the absorbing portion into the flat portions 24 extending in the width direction of the absorber 13. In the example illustrated in FIG. 11, the recessed portions 21r are arranged at regular intervals, and the flat portions 24 are each positioned between the recessed portions 21r. The adjacent flat portions 24 are separated from each other by the recessed portion 21r. In the example illustrated in FIG. 11, in order to suppress the sticky feeling, each flat portion 24 needs to have a certain size as is the case with Embodiment 1. Specifically, the optimal size of the flat portion 24 is determined based on the diameter R1 of the maximum inscribed circle (in FIG. 11, depicted by an alternate long and short dash line) inscribed in the flat portion 24 sandwiched between the two recessed portions 21r adjacent to each other along an arranging direction of the recessed portions 21r and the width L1 of the recessed portion 21r along the radial direction of the maximum inscribed circle. The relation between the diameter R1 and the width L1 is similar to the corresponding relation in Embodiment 1.

Furthermore, in the present invention, the groove portions 21 may be formed to extend parallel to the virtual line P or formed like a lattice along with recessed portions 21r extending parallel to the virtual line P (or no such recessed portions 21r are provided). Moreover, in the present invention, the groove portions 21 may be configured such that the recessed portions 21r are not arranged at regular intervals.

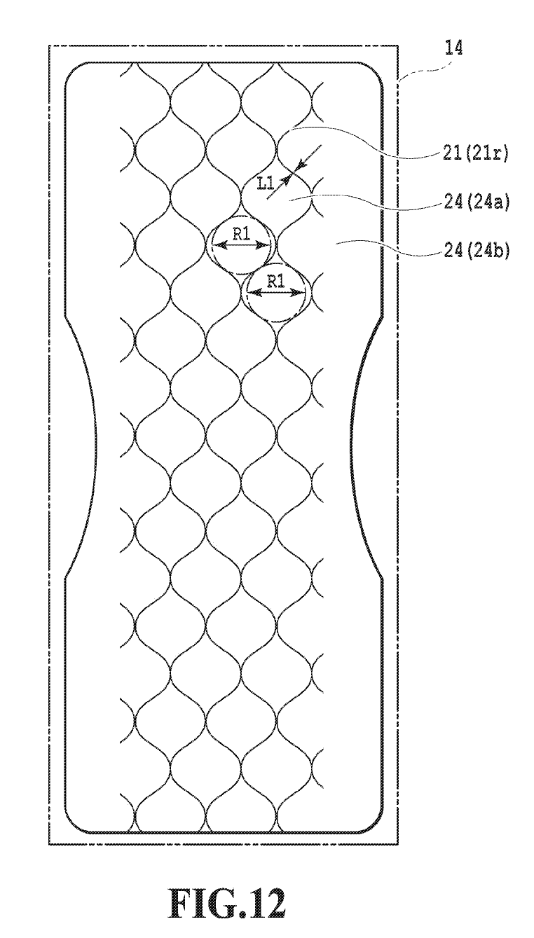

FIG. 12 is a partial top view illustrating an example of a flat portion pattern formed by arranging wavelike recessed portions 21r (groove portions 21) in the absorbing portion. As depicted in FIG. 12, the recessed portions 21r may be curves. In the example illustrated in FIG. 12, the recessed portions 21r are obtained by forming sine curve-like lines along the front-back direction of the absorber 13 and arranging a plurality of the sine curve-like lines in the width direction of the absorber 13. Furthermore, as depicted in FIG. 12, the recessed portions 21r are formed by arranging the sine curves such that the adjacent sine curves are in contact with each other while being out of phase with each other by 180 degrees. As depicted in FIG. 12, parts of the flat portion 24 that are enclosed by the wavelike recessed portions 21r are first flat portions 24a, and parts of the flat portion 24 that are not enclosed by the wavelike recessed portions 21r are second flat portions 24b.

The flat portion pattern depicted in FIG. 12 also enables the sticky feeling to be suppressed in a case where a condition similar to the condition in Embodiment 1 is met by the relation between the diameter R1 of the maximum inscribed circle inscribed in each first flat portion 24a enclosed by the recessed portions 21r and the width L1 of the recessed portion 21r along the radial direction of the maximum inscribed circle.

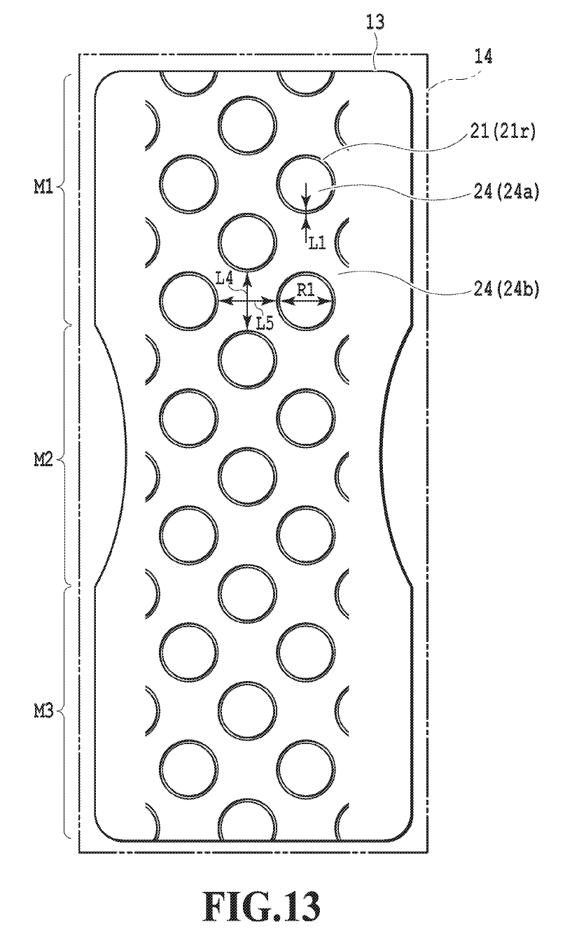

FIG. 13 is a partial top view illustrating an example of a flat portion pattern formed by arranging circular recessed portions 21r (groove portions 21) at regular intervals in the absorbing portion. As depicted in FIG. 13, the first flat portions 24a each enclosed by the corresponding recessed portion 21r may be scattered like islands. In this case, the diameter R1 of the maximum inscribed circle inscribed in each first flat portion 24a enclosed by the recessed portion 21r is set to meet the relation in which the diameter R1 is at least five times as large as the width L1 of the recessed portion 21r along the radial direction of the maximum inscribed circle. Moreover, the second flat portion 24b positioned between a plurality of the first flat portions 24a is generally flat and thus serves as the main absorbing area for the discharged body fluid. In a case where the adjacent first flat portions 24a are in proximity to each other, the skin is contacted mostly by the first flat portion 24a. Consequently, the effect of this pattern is similar to the effect of the pattern in which the first flat portions 24a are separated from each other by the recessed portions 21r as depicted in FIG. 4, FIG. 11, and FIG. 12. On the other hand, in a case where a certain separation distance is present between the adjacent first flat portions 24a, the skin is contacted both by the first flat portions 24a and by the second flat portion 24b. The second flat portion 24b between the first flat portions 24a produces an effect similar to the effect of the first flat portions 24a. Here, for the distance between the recessed portion 21r enclosing the first flat portion 24a and the adjacent recessed portion 21r, a distance in the longitudinal direction of the absorbing portion of the diaper 10 (the up-down direction in FIG. 13) is denoted by L4, and a distance in a direction orthogonal to the longitudinal direction (the lateral direction in FIG. 13) is denoted by L5. In this case, in a case where the diameter R1 of the maximum inscribed circle inscribed in each first flat portion 24a enclosed by the recessed portion 21r is 10 mm or more and 30 mm or less, each of the distances L4, L5 is preferably 5 mm or more and 30 mm or less.

In FIG. 13, the island-like first flat portions 24a are composed of a plurality of circles with the same size. However, in the present invention, the island-like first flat portions 24a may be configured by combining with a plurality of circles with different sizes. Furthermore, in the present invention, the island-like first flat portions 24a are not limited to circles. Various shapes such as a heart shape which are excellent in design are applicable. That is, any shape may be used so long as the area enclosed by the recessed portion 21r is flat.

Additionally, the island-like first flat portions 24amay be densely provided in the groin portion M2 and the back body portion M3 rather than being evenly arranged all over the absorbing portion.

In the above-described embodiments, the case has been described where all of the groove portions 21 are composed of the recessed portion 21r. However, the present invention is effective for suppressing a possible sticky feeling even in a case where all of the groove portions 21 are entirely not composed of the recessed portion 21r, that is, parts of the groove portions 21 are composed of the recessed portion 21r.

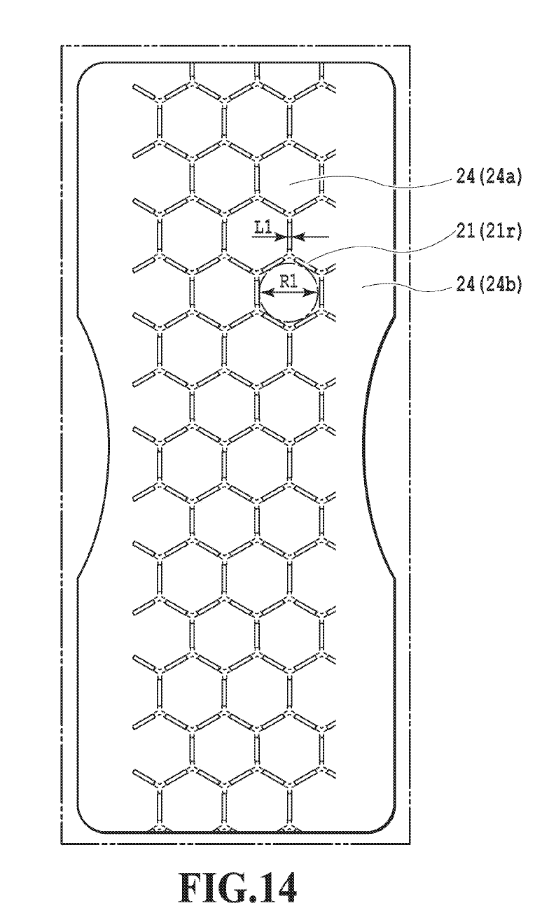

FIG. 14 is a partial top view illustrating an example of a flat portion pattern formed by arranging, in the absorbing portion, discontinuous recessed portions 21r shaped like regular hexagons. In the present embodiment, as depicted in FIG. 14, the groove portion 21 is partly composed of the recessed portion 21r. In FIG. 14, the recessed portion 21r of the groove portion 21 is depicted by a solid line, and parts of the groove portion 21 in which no recessed portions 21r are formed are depicted by dashed lines, for convenience of description. As depicted in FIG. 14, the flat portion 24 has two types of flat portions, the first flat portion 24a that is enclosed by the groove portion 21 and the second flat portion 24b that is not enclosed by the groove portion 21.

The flat portion pattern may be shaped like a honeycomb as depicted in FIG. 14. Furthermore, the first flat portion 24a need not be entirely enclosed by the recessed portion 21r as described above, but maybe partly enclosed by the recessed portion 21r. In a case where the first flat portion 24a is partly enclosed by the recessed portion 21r, in a case where at least 50% or more of the outer periphery of the first flat portion 24a is enclosed by the recessed portion 21r, the shape of the first flat portion 24a is easily maintained even after the body fluid is absorbed.

The flat portion pattern depicted in FIG. 14 also enables the sticky feeling to be suppressed in a case where a condition similar to the condition in Embodiment 1 is met by the relation between the diameter R1 of the maximum inscribed circle inscribed in each first flat portion 24a enclosed by the groove portion 21 and the width L1 of the groove portion 21 along the radial direction of the maximum inscribed circle.

In the present invention, for the compressed portion 22, not only circular dots but also various other shapes such as a semicircle, a quadrilateral, a triangle, star shape may be used.

In addition, the diaper may be formed by wrapping the absorber 13 with a hydrophilic sheet (for example, a core wrap) and then disposing the top sheet 14 on the absorber 13 instead of disposing the top sheet 14 directly on the absorber 13. Furthermore, a liquid diffusion sheet that improves liquid diffusability may be provided between the top sheet 14 and the hydrophilic sheet or between the hydrophilic sheet and the absorber 13. The liquid diffusion sheet allows the body fluid to diffuse easily. Alternatively, the hydrophilic sheet may be attached to the absorber 13 so as to wrap the absorber 13 or simply placed on each of the front and back surfaces of the absorber 13 without wrapping the ends of the absorber 13.

The structure of the diaper 10 to which the present invention is directed is not limited to the above-described pull-up type. The diaper 10 may have any configuration so long as the diaper 10 includes the configuration of an absorbent article defined in the claims. For example, the present invention is applicable to well-known unfolding disposable diapers and incontinence pads.

In addition, the present invention is not limited to diapers for infants but is applicable to various absorbent articles such as diapers for adults and incontinence pads which are adapted for the growth stage, body type, sex, and the like of the wearer.

Furthermore, the above-described embodiments and variations thereof may be combined with one another.

### REFERENCE SIGNS LIST

10 Diaper
10F Front body area
10R Back body area
10C Groin area
10W Waist opening
10L Leg opening
10J Closed portion
10T Disposal tape
11 Cover sheet
11A Pad cover sheet
11B Inner cover sheet
11C Outer cover sheet
12 Back sheet (back surface sheet)
13 Absorber
14 Top sheet (front surface sheet)
17 Rubber thread
18 Side sheet
19 Rubber thread
21 Groove portion
21r Recessed portion
21a First recessed portion
21b Second recessed portion
22 Compressed portion
24 Flat portion
24a First flat portion
24b Second flat portion

1. An absorbent article having a liquid-permeable front surface sheet, a liquid-impermeable back surface sheet, and an absorber disposed between the front surface sheet and the back surface sheet, the absorbent article comprising:
a groove portion entirely or partly composed of a recessed portion, the recessed portion being formed on the front surface sheet side of the absorbent article so as to integrate the front surface sheet and the absorber together; and
a flat portion enclosed by the groove portion,
wherein a diameter of a maximum inscribed circle inscribed in the flat portion is at least five times and at most 20 times as large as a width of the groove portion along a radial direction of the maximum inscribed circle.

2. The absorbent article according to claim 1, wherein the recessed portion encloses at least 50% of an outer periphery of the flat portion.

3. The absorbent article according to claim 1, wherein the recessed portion is formed by compressing the front surface sheet and the absorber together, and a depth of the recessed portion is at least half of a thickness of the flat portion.

4. The absorbent article according to claim 1, wherein the groove portions are shaped like a lattice.

5. An absorbent article having a liquid-permeable front surface sheet, a liquid-impermeable back surface sheet, and an absorber disposed between the front surface sheet and the back surface sheet, the absorbent article comprising:
a groove portion entirely or partly composed of a recessed portion, the recessed portion being formed and arranged on the front surface sheet side of the absorbent article so as to integrate the front surface sheet and the absorber together; and
a flat portion positioned between the groove portions,
wherein a diameter of a maximum inscribed circle inscribed in the flat portion sandwiched between the two adjacent groove portions is at least five times and at most 20 times as large as a width of the groove portion along a radial direction of the maximum inscribed circle.

6. The absorbent article according to claim 5, wherein the groove portions are arranged at regular intervals.

7. The absorbent article according to claim 5, wherein an amount of SAP per unit area in the absorber is at least 0.01 g/cm2.

8. The absorbent article according to claim 5, wherein the absorbent article is a disposable diaper.

9. The absorbent article according to claim 1, wherein an amount of SAP per unit area in the absorber is at least 0.01 g/cm².

10. The absorbent article according to claim 1, wherein the absorbent article is a disposable diaper.
